(19) United States
(12) Patent Application Publication
(10) Pub. No.: US 2024/0024357 A1
(43) Pub. Date: Jan. 25, 2024

(54) METHODS OF PREPARING IRON COMPLEXES

(71) Applicants: Chien-Chin WU, Wilmington, DE (US); LG Bionano, LLC, Wilmington, DE (US)

(72) Inventor: Chien-Chin Wu, Wilmington, DE (US)

(21) Appl. No.: 18/023,086

(22) PCT Filed: Sep. 29, 2021
(86) PCT No.: PCT/US2021/052571

§ 371 Date: Feb. 24, 2023

(30) Foreign Application Priority Data
Sep. 29, 2020 (CN) 202011055787.3
Sep. 29, 2020 (CN) 202011055789.2

(51) Int. Cl. A61K 33/26; A61K 47/26; A61K 47/54

(52) U.S. Cl. CPC A61K 33/26; A61K 47/26; A61K 47/549

(57) ABSTRACT

A method of preparing an iron hydroxide product is provided. The method includes the steps of: adding a first base solution to a solution of a ferric salt to obtain Mixture A having a pH value of 2.7-2.8, adding a second base solution to Mixture A to prepare a crude iron hydroxide suspension having a pH value of 2.8-3.8, and adding a third base solution to adjust the pH of the crude iron hydroxide suspension to 5-9, followed by purification and concentration, thereby obtaining a purified polynuclear iron hydroxide suspension containing polynuclear iron hydroxide. Also provided are nano iron complexes, e.g., iron hydroxide carbohydrate complex and their preparation methods.

### CROSS REFERENCES TO RELATED APPLICATIONS

This application claims the benefit of priority based on Chinese Application Nos. 202011055789.2 and 202011055787.3, both filed Sep. 29, 2020. The contents and disclosures of both applications are incorporated herein by reference in their entirety.

### BACKGROUND

Iron plays a key role in human oxygen delivery. Iron deficiency is the most common nutritional deficiency in humans, resulting in anemia. Patients with advanced renal failure have serious iron deficiency anemia. Their survival rates are closely related to iron supply.

Iron injection is the first choice for the care of such patients. Regular iron supplements, e.g., ionic irons or small molecular irons, have high oxidation potential, causing oxidative damage to organs. In the past three decades, carbohydrate-coated iron hydroxide nanoparticles have become the mainstream source in iron injection. Among them, iron hydroxide sucrose complex nanoparticles are the first choice for commercial iron injection because of their fast onset and minimal side effects.

To optimize the safety and efficacy, the iron sucrose complex nanoparticles each should have an appropriate particle size. If the nanoparticles are too small, iron will release at a fast rate, leading to serious oxidative damages. If the nanoparticles are too large, the slow onset will likely cause severe allergic side effects.

It has been found that useful iron hydroxide sucrose complex nanoparticles each have a molecular weight in the range 32000-60000 Daltons. As such, this molecular weight range is required of iron nanoparticle products by US Pharmacopeia.

Iron hydroxide sucrose complex includes polymeric nanoparticles. Their stability and particle size are closely related to process conditions such as temperature, reaction rate, acid-base conditions, etc. Preparation remains challenging. Current commercial iron sucrose complexes have been prepared by low-temperature processes requiring expensive explosive-proof equipment. As indicated in certain patents, when the process temperature is 20° C. or higher, the nano iron sucrose complex thus prepared has a molecular weight exceeding 80000 Daltons, rendering the product useless.

The iron release rate is another quality-defining feature for nano iron sucrose complex. If released too fast, it will cause oxidative damages as a side effect. Vifor Pharma Group, a manufacturer of nano iron sucrose injectable products, requires as a quality control management that the iron release rate should be within 20 minutes under the acidic condition of vitamin C Currently, a reliable preparation process has not been found in any patents covering sucrose-coated iron hydroxide that meets this important quality requirement.

Chinese Application Publication CN1853729A discloses a preparation method of polynuclear iron hydroxide sucrose complex. The polynuclear iron hydroxide component is prepared at a low temperature of 5-20° C. It then chelates with sucrose at a high temperature of 106-125° C. to afford a product having a molecular weight outside the range required by USP.

Chinese Application Publication CN109893540A discloses a preparation method of iron sucrose complex solution with low heavy metal content. The specific steps are as follows: 1. to a 50-80° C. sodium carbonate aqueous solution, adding ferric chloride under agitation, allowing it to react until the solution is black, removing the sediment by filtration; 2. cooling the filtrate to 0-5° C., stirring for 4-8 h, and adding dropwise a sodium carbonate solution until the pH of the reaction mixture reaches pH 7-9; and 3. adding sodium hydroxide to iron hydroxide colloid, adjusting the pH value of the reaction mixture to no less than 10, and adding sucrose to the mixture and heating the reaction solution until boiling to obtain nano iron sucrose complex. Problems with the process described in CN109893540A include long processing time and low temperature, thus a costly process. Further, the rapid formation of iron complex made it difficult to control turbidity point, an important quality indicator.

Chinese patent CN103059072A discloses an environmentally friendly method for preparing iron sucrose complex. The steps are as follows: (1) preparing a 0.5-5 wt % FeCl₃·6H₂O solution and a Na₂CO₃ solution, adding the Na₂CO₃ solution to the FeCl₃·6H₂O solution at 0° C.-30° C. with a peristaltic pump during a feeding time of stirring for 1 hour and centrifuging the resulting suspension to obtain a Fe(OH)₃ cake, repeating mixing/centrifugation/washing with purified water for 4 times to obtain a purified Fe(OH)₃ cake, and determining the content of iron in the Fe(OH)₃ cake, the ratio of FeCl₃·6H₂O to Na₂CO₃ being 1:0.55-0.65; (2) adding sucrose to Fe(OH)₃ thus obtained at a ratio of Fe:sucrose=1:13.5-16.5, heating the resultant mixture to 85-140° C. and pH 8-13 for 2-18 h, and cooling the mixture to obtain a nano iron sucrose complex; and (3) treating the above complex with D301 anion exchange resin and then D113 cation exchange resin, adjusting the pH value to and filtering through a 0.8 μm ultrafilter to obtain a final nano iron sucrose product. The process described in CN103059072A is costly due to the required low temperature, high pressure, and use of ion exchange resin for purification.

Other known iron products include ferric citrate, ferric pyrophosphate citrate, and ferric gluconate. See, e.g., U.S. Pat. Nos. 9,624,155, 7,816,404, 7,767,851, and 7,005,531. These products each have deficiencies such as a low water solubility, a low iron content, and inefficient process, which is greatly limiting their application.

There is a need to develop a cost-effective method of preparing an iron product suitable for pharmaceutical and nutritional use.

### SUMMARY OF THE INVENTION

To address the problems of current low temperature processes mentioned above, this invention provides efficient methods of preparing at ambient conditions iron hydroxide complexes that have superior properties including high water solubility, high iron content, and great bioavailability.

Accordingly, one aspect of the invention relates to a method of preparing an iron hydroxide product. The method includes the steps of: (1) adding a first base solution to a solution of a ferric salt to obtain Mixture A having a pH value of 2.7-2.8, (2) adding a second base solution to Mixture A to prepare a crude iron hydroxide suspension having a pH value of 2.8-3.8, and (3) adding a third base solution to adjust the pH of the crude iron hydroxide suspension to 4.5-9.5 (e.g., 5-9), followed by purification and concentration, thereby obtaining a purified polynuclear iron hydroxide suspension containing polynuclear iron hydroxide as an exemplary product of this invention.

Purification is performed following traditional methods, e.g., by washing with water and then concentrating by removing water, to obtain an iron hydroxide suspension having an iron hydroxide concentration of 3 wt % to 16 wt %. After the purification, the chlorine content falls below 1% (e.g., below 0.1% and below 0.025%). The purification step also removes free Fe³⁺ and Fe²⁺, which contribute to iron oxidative damages to organs of a patient. The free iron cations are also the source of undesirable metallic taste of a final product, e.g., an oral formulation.

It is preferred that, before adding the second base, Mixture A is allowed to equilibrate for 1 minute to 15 minutes and, before adding the third base, the crude iron hydroxide suspension is allowed to equilibrate for 2 minutes to 60 minutes, both at an ambient temperature, e.g., 20° C.-30° C. and 25° C.

Typically, each of the first base solution, the second base solution, and the third base solution, independently, is added at a temperature of 15° C.-50° C. (e.g., 20-20-30° C., and 22-27° C.). The first base solution, the second base solution, and the third base solution, independently, can be an aqueous solution of a carbonate salt, e.g., NaHCO₃, Na₂CO₃, (NH₄)₂CO₃, and K₂CO₃. The preferred solution is an aqueous Na₂CO₃ solution having a mass percentage of 1% to 25% (e.g., 3% to 20%, 5% to 15%, and 10%). Further, the first, second, and third base solutions can be the same or different.

Suitable ferric salts include Fe₂(SO₄)₃, Fe(NO₃)₃, FeCl₃, and their hydrates. A preferred ferric salt is FeCl₃ (e.g., FeCl₃·6H₂O) having a mass percentage of 5% to 60%, preferably 15% to 25%.

In some embodiments, the method further contains the steps of: (i) mixing the purified polynuclear iron hydroxide suspension and a carbohydrate to obtain a carbohydrate mixture, (ii) adjusting the pH value of the carbohydrate mixture to 7.5-13 or 9.5-13.5 (e.g., 10-13.5), and (iii) heating the pH-adjusted carbohydrate mixture to a temperature of 60° C.-125° C. (preferably 75-95° C. and more preferably 80-95° C.), thereby producing an iron hydroxide-carbohydrate complex suspension, in which the mass ratio between iron and carbohydrate is (1-1100):100, and the iron hydroxide product is the iron hydroxide carbohydrate complex.

Exemplary carbohydrates include monosaccharides, disaccharides, oligosaccharides, polysaccharides, hydrolyzed polysaccharides, and any combinations thereof. In a preferred embodiment, the carbohydrate is sucrose and the mass ratio between Fe³⁺ and sucrose is 1:(10-20), e.g., 1:(13-17).

Optionally, the pH value of the carbohydrate mixture is adjusted by adding a fourth base that is a hydroxide solution selected from the group consisting of a NH₄OH solution, a KOH solution, and a NaOH solution, and the hydroxide solution has a mass percentage of 5% to 50%, preferably 10% to 25%.

In an example, the pH value of the carbohydrate mixture is adjusted to 9.5-13.5 (e.g., 10-13.5) and the pH-adjusted carbohydrate mixture is heated at 80° C.-125° C. (e.g., 85° C.-95° C.) for 1 hour to 50 hours.

After the complex is formed, its pH value is optionally adjusted using a pH modifier to 5.5-11.1, 10.5-11.2, or 6.5-7.5. Suitable pH modifiers include HCl, NaOH, citric acid, oxalic acid, fumaric acid, tartaric acid, succinic acid, malic acid, ascorbic acid, phosphoric acid, pyrophosphoric acid, and glycophosphoric acid.

In another example, the pH value of the carbohydrate mixture is adjusted to 7.5-13 (e.g., 9-12.5), the pH-adjusted carbohydrate mixture is heated at 65° C.-121° C., preferably 80° C.-95° C., for 0.2 hours to 30 hours, and the mass ratio between iron and carbohydrate is (1-264):24.

The iron hydroxide-carbohydrate complex thus prepare has properties suitable for human consumption in treating iron deficiency related disorders. Desirable properties include one or more of the following features: a weight average molecular weight of 30000-60000, a reaction rate T75 against ascorbic acid of less than 35 minutes, containing no free ferric ions, a water solubility of 20 wt % or more (e.g., 50% or more, 20-50 wt %, and 35 wt %), an iron content by dry weight of 10%-47% (e.g., 15-47%), and a chloride ion content of less than 1% (e.g., less than 0.1%).

In other embodiments, the method of this invention further includes the steps of: (i) mixing the purified polynuclear iron hydroxide suspension with citric acid, a citrate salt, or combination thereof to obtain a citrate mixture, and (ii) heating the citrate mixture at a temperature of 40° C.-105° C. (e.g., 45-95° C. and 55-65° C.) for 2 minutes to 10 hours (e.g., 2-180 minutes and 5-30 minutes) thereby producing an ferric citrate complex suspension containing iron hydroxide-citrate complex, in which the molar ratio between iron and citrate being 1:(0.3-5), preferable 1:(0.6-1.5), and the iron hydroxide product is an iron hydroxide-citrate complex. As compared to current products available in the market, the iron hydroxide-citrate complex surprisingly has superior properties, e.g., a water solubility of 20 wt % or greater (e.g., 50 wt % or greater), a high iron content (e.g., by dry weight 5%-35% and 12%-25%), and absence of free ferric ions.

In still other embodiments, the method further include the of: (a) mixing the purified polynuclear iron hydroxide suspension and a solution containing (i) citric acid or a citrate salt and (ii) pyrophosphoric acid or a pyrophosphate salt to obtain a pyrophosphate mixture, and (b) heating the pyrophosphate mixture at a temperature of 40° C.-105° C. (e.g., 55-65° C.) for 5 minutes to 10 hours (e.g., 25-55 minutes), thereby producing a ferric citrate pyrophosphate suspension, in which the molar ratio of iron:citrate:pyrophosphate is 1:(0.3-3):(0.3-3) and the iron hydroxide product is an iron hydroxide-citrate-pyrophosphate complex that has a water solubility of 20 wt % or greater (e.g., 50 wt % or greater) and contains by dry weight iron 3%-35% (e.g., 5-%).

In yet other embodiments, the method further include the steps of: (a) mixing the purified polynuclear iron hydroxide suspension with a carboxylated carbohydrate to obtain a carboxylated carbohydrate mixture, and (b) heating the carboxylated carbohydrate mixture at a temperature of 50° C.-125° C. (e.g., 65-125° C., 55-75° C., and 65-75° C.) for 5 minutes to 10 hours (e.g., 25-55 minutes), thereby producing a ferric carboxylated carbohydrate suspension, in which the molar ratio between iron and the carboxylated carbohydrate is 1:(0.3-5), preferable 1:(0.5-1.5), and the iron hydroxide product is an iron hydroxide-carboxylated carbohydrate complex. Exemplary carboxylated carbohydrates are gluconate and other carboxylated di-saccharides, oligosaccharides, and polysaccharides.

Further, the method of this invention includes the additional steps of: (a) mixing the purified polynuclear iron hydroxide suspension with a multivalent anion to obtain a multivalent anion mixture, (b) adjusting the pH value of the multivalent anion mixture to 2-13, preferably 3-9, and (c) heating the pH-adjusted multivalent anion mixture to a temperature of 40° C.-125° C., preferably 50° C.-95° C., thereby producing a nano ferric complex suspension, in which the mass ratio between iron and the multivalent anion is (1-1100):100 and the iron hydroxide product is the nano ferric complex.

Any complex suspension thus prepared can be dried by a conventional drying method, e.g., spray drying. Either in a liquid form or a dry form, the iron hydroxide-carbohydrate complex can be formulated into drops, oral liquid, suspension, injection, powder, capsule, tablet, or lozenge for treating iron deficiency anemia in humans or animals.

Also within the scope of this invention are iron hydroxide products prepared from any method described above. These products include pharmaceutical compositions and nutraceutical compositions containing an iron hydroxide product of this invention and a pharmaceutically or nutraceutically acceptable carrier.

Still within the scope of this invention is a method of treating an iron deficiency-related disorder or hyperphosphatemia by administering to a subject in need thereof a pharmaceutically effective amount of an iron hydroxide product described above.

The details of several embodiments of the present invention are set forth in the description below. Other features, objects, and advantages of the invention will be apparent from the description, the drawings, and also from the appended claims.

### BRIEF DESCRIPTION OF THE DRAWINGS

FIG. 1 is a gel permeation chromatograph (GPC) for determining the molecular weight of the iron hydroxide carbohydrate complex prepared in Example 1 below.

FIG. 2 is a structural diagram for a representative nanoparticle of an iron hydroxide carbohydrate complex of this invention.

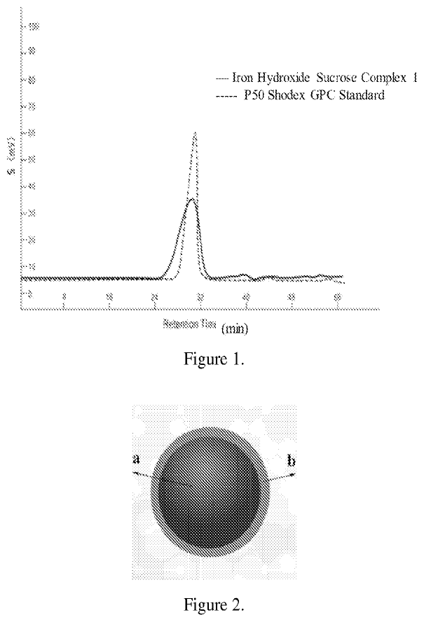

### DETAILED DESCRIPTION OF THE INVENTION

One objective of the invention is to solve the problems of current low temperature process, which is slow and costly as described in the background section above. Further, current preparation at the room temperature results in an iron hydroxide-sucrose complex with an undesirable high molecular weight.

Another objective of the invention is to solve the problem of high levels of ferric and chloride ions that remain in iron hydroxide carbohydrate complex products.

Accordingly, the invention provides cost effective methods of preparing nano iron hydroxide complexes.

The first method of this invention includes the following steps.
(1) adding a first base aqueous solution to an iron salt solution in water and mixing evenly until the pH value reaches 2.7-2.8,
(2) adding the first base aqueous solution the mixture obtained in step (1) above and mixing it evenly to obtain a reaction mixture having a pH value of 2.8-3.8 to obtain a crude iron hydroxide suspension,
(3) adding the first base aqueous solution to the crude iron hydroxide suspension and mixing it evenly to obtain an iron hydroxide suspension having a pH value of 4.5-9,
(4) collecting iron hydroxide particles from the suspension, followed by purification and concentration to obtain a purified polynuclear iron hydroxide wet cake.

The second method of this invention includes the step of: adding a carbohydrate to the purified polynuclear iron hydroxide and mixing evenly to obtain a mixture, adjusting the pH value of the mixture to 9.5-13.5 with a step 2 base aqueous solution, and heating the pH-adjusted mixture for 1-50 hours at 85° C.-125° C. to obtain an iron hydroxide carbohydrate complex.

A third method of this invention includes the following steps: adding a carbohydrate to the polynuclear iron hydroxide and mixing them evenly to obtain Mixture B, adjusting the pH value of Mixture B to 7.5-13 with a step 2 base solution, and then heating at 60° C.-125° C. for 0.2-30 h to obtain an iron hydroxide carbohydrate complex, in which the ratio of iron and carbohydrate is (1-1100):100.

A fourth method of this invention contains the steps of: mixing the purified polynuclear iron hydroxide suspension with a multivalent anion or a carboxylated carbohydrate at a temperature of 45° C.-125° C. (e.g., 55-95° C., 75-95° C., 45-65° C., and for 2 minutes to 6 hours, thereby producing a nano iron hydroxide complex suspension containing iron hydroxide-multivalent anion complex or iron hydroxide-carboxylated carbohydrate complex.

Suitable multivalent anions include citric acid, tartaric acid, succinic acid, fumaric acid, malic acid, glyceryl phosphoric acid, any salt thereof, and any combination thereof. These multivariant anions can be used in combination with pyrophosphate.

Exemplary carboxylated carbohydrates are gluconate and other carboxylated di-saccharides and polysaccharides. Gluconic acid or any water-soluble gluconate salt (e.g., alkali-D-gluconate such as sodium-D-gluconate) can be used in the preparation.

The advantages of the above methods are summarized below.
(1) The iron hydroxide carbohydrate complexes are prepared at room temperature (e.g., 15-40° C., 20-35° C., 22-27° C., and 25° C.) thus avoiding low-temperature and high-cost routes. The temperature is also accurately controlled due to the three-step pH titration process for preparing the Fe(OH)₃ suspension.
(2) The methods are low-chloride processes, which also avoid pollution by heavy metals. The chloride content is less than 0.1% by weight of the complexes.
(3) The iron hydroxide carbohydrate complexes thus prepared has no residual free ferric iron and thus minimize potential oxidative damages to human bodies. With a high iron content, the complexes each have an iron hydroxide core and a carbohydrate shell coating the core, at an appropriate carbohydrate:iron ratio. No residual free trivalent iron is detected in these complexes.
(4) The iron hydroxide-sucrose complex is an example of the iron hydroxide carbohydrate complexes prepared by the invention at room temperature. Its weight average molecular weight is 30000-60000 Daltons, a desirable molecular weight range to provide fast iron release and fast onset with a high safety margin.
(5) The iron hydroxide sucrose complexes each have a reaction rate T75 against ascorbic acid of 35 min or less. It meets the required quality for quick onset and safety.
(6) The iron hydroxide sucrose complexes can be basic (pH greater than 7) or neutral (pH around 7). They are stable and can be sterilized at a high temperature.
(7) The iron hydroxide carbohydrate complexes can be in a liquid form or a solid form, conveniently being formulated into any liquid or solid dosage forms.
(8) The processes, unlike certain known processes, do not require an organic solvent, avoiding pollution by an organic residue.
(9) The iron hydroxide complexes each have a high water solubility (e.g., 20 wt % or greater and 50 wt % or greater). They are suitable for the development of high-dose liquid dosage forms (such as drops with 10 wt % iron).
(10) The methods can be performed under mild conditions without refrigeration, pressurization or explosion-proof equipment, making them easy for industrial implementation. And there are few impurities and high bioavailability. It can be used to develop better iron supplement products for the treatment of iron deficiency anemia or phosphate removal for kidney dialysis patients.

The following specific embodiments further illustrate the methods of the invention. But they should not be understood as limiting the invention. Without departing from the essence of the invention, the modification and replacement of the methods, steps or conditions of the invention fall within the scope of the invention.

### Iron Hydroxide Carbohydrate Complexes

In both the first and second methods described above, the first base aqueous solution can be an aqueous solution of a carbonate or bicarbonate salt, e.g., NaHCO₃, Na₂CO₃, (NH₄)₂CO₃, and K₂CO₃. The preferred base is Na₂CO₃. Its mass percentage in the aqueous solution is typically at 5%-25%, preferably 10%-15%. Other steps are the same as in embodiment 1. Carbonate or bicarbonate salts include their anhydrous and hydrate forms. Examples are Na₂CO₃·H₂O, Na₂CO₃·7H₂O, Na₂CO₃·10H₂O.

The iron salt solution can be an aqueous solution of Fe₂(SO₄)₃, Fe(NO₃)₃, FeCl₃, or any combination thereof. Iron salts include their anhydrous and hydrate forms, e.g., FeCl₃·6H₂O and Fe(NO₃)₃·9H₂O. The preferred salt is FeCl₃ including FeCl₃·6H₂O. The mass percentage of the iron salt solution can be 5%-60% (e.g., 15%-25%).

A preferred carbohydrate for the first method is sucrose. The mass ratio of Fe³⁺ to sucrose in the mixture is 1:(10-20), preferably 1:(13-17). A preferred carbohydrate for the second method includes a monosaccharide, a disaccharide, an oligosaccharide, a polysaccharide, a polysaccharide hydrolyzed syrup, and any combination thereof.

The step 2 base aqueous solution can be an aqueous solution of hydroxide compounds, e.g., NH₄OH, KOH, and NaOH, preferably NaOH at a mass percentage in the aqueous solution of 5%-50% (e.g., 10%-25%).

For the first method, the pH value of the iron hydroxide carbohydrate complex obtained in step 2 can be adjusted to 5.5-11.1 (e.g., 10.5-11.1 and 6.5-7.5) using a pH value modifier. Suitable examples of a pH modifier include HCl, NaOH, and an organic or inorganic acid selected from the group consisting of citric acid, oxalic acid, fumaric acid, tartaric acid, succinic acid, malic acid, ascorbic acid, phosphoric acid, pyrophosphoric acid, and glycophosphoric acid. For the second method, a carbohydrate is added to the polynuclear iron hydroxide and mixed evenly to obtain mixture B followed by adjusting the pH value of mixture B to 9.5-12.5 and reacting at 65° C.-95° C. for 0.2 h-30 h to obtain an iron hydroxide carbohydrate complex with the iron/sugar ratio of (1-264):24.

The iron hydroxide carbohydrate complexes thus prepared are either in a liquid form or a solid form. For solid use, a drying step is included such as spray drying. The iron hydroxide carbohydrate complex thus prepared can be formulated into drops, oral liquids, injections, powders, capsules, suspension dosage forms or tablets for the treatment of iron deficiency anemia in humans or animals.

Some iron hydroxide carbohydrate complexes (e.g., an iron hydroxide sucrose complex prepared by the first method) have one of the following preferred features: a weight average molecular weight of 30000-60000 Daltons, a reaction rate T75 against ascorbic acid of 35 minutes of less, no residual free ferric iron, a high stability under high pH or neutral conditions, and capability of being sterilized at a high temperature. Other iron hydroxide carbohydrate complexes (e.g., prepared by the second method) have a water solubility of 20%-50%, an iron content by dry weight of 15%-47%, and a chloride ion content of less than 1% (e.g., less than 0.1% and less than 0.025%).

Also within the scope of this invention are iron hydroxide products prepared by any of the methods described above. As illustrated by FIG. 2, each of the iron hydroxide products has a polynuclear iron hydroxide core and a shell formed of a carbohydrate, a multivalent anion, or a carboxylated carbohydrate.

Still within the scope of the invention is a method of treating an iron deficiency-related disorder (e.g., anemia) by administering an effective amount of an iron hydroxide product or a pharmaceutical composition containing same to a subject in need thereof.

The term “treating” refers to application or administration of the compound to a subject with the purpose to cure, alleviate, relieve, alter, remedy, improve, or affect the disease, the symptom, or the predisposition. “An effective amount” refers to the amount of the products which is required to confer the desired effect on the subject. Effective amounts vary, as recognized by those skilled in the art, depending on route of administration, excipient usage, and the possibility of co-usage with other therapeutic treatments. Dosage levels of an iron hydroxide product of this invention are of the order of 1 mg/day to 200 mg/day (e.g., 150 mg/day, 45 mg/day, 15 mg/day, 2 mg/day to 45 mg/day, 3 mg/day to 30 mg/day, and 5 mg/day to 25 mg/day). The specific dose level for a particular patient will depend upon a number of factors including age, body weight, general health, sex, diet, time of administration, rate of excretion, and the severity of iron deficiency.

The term “carbohydrate” refers to aldehyde or ketone compounds substituted with multiple hydroxyl groups, of the general formula (CH₂O)ₙ, in which n is 3-300. Carbohydrates include monosaccharide (n=3-10), disaccharide (n=8-14, e.g., 12, having two monosaccharide units), oligosaccharide (n=15-59, i.e., having 3-9 monosaccharide units), as well as polysaccharide (i.e., having 10 or more monosaccharide units). Monosaccharides cannot be broken down to simpler sugars by hydrolysis. They constitute the building blocks of disaccharides, oligosaccharides and polysaccharides. Examples include glyceraldehyde, dihydroxyacetone, erythrose, threose, arabinose, ribose, xylose, ribulose, xylulose, glucose (dextrose), fructose, galactose, ribose, allose, altrose, gulose, idose, mannose, talose, psicose, sorbose, tagatose, mannoheptulose, sedoheptulose, 2-keto-3-deoxy-manno-octonate, and sialose. Examples of a disaccharide include sucrose, maltose, isomaltose, lactose, trehalose, cellobiose, chitobiose, rutinose, and rutinulose. The term “carboxylated carbohydrate” refers to a carbohydrate containing a carboxyl group (—COOH or —COO—). They can be prepared by oxidizing a corresponding original carbohydrate.

The term “water solubility” refers to colloidal solubility in water, i.e., the maximal concentration of dispersed nanoparticles that coexist with agglomerates in equilibrium. See Doblas, et al., Nano Lett. 19, 5246-52 (2019). The water solubility of an iron product of this invention is the maximal concentration that the iron product is evenly dispersed in water as a clear liquid without precipitation or cloudiness.

Without further elaboration, it is believed that one skilled in the art can, based on the above description, utilize the present invention to its fullest extent. The following examples are to be construed as merely illustrative and not limitative of the remainder of the disclosure in any way whatsoever. All publications cited herein are hereby incorporated by reference in their entirety.

Set forth below are examples illustrating methods of preparing iron hydroxide complexes and efficacy evaluation thereof.

### EXAMPLES

### Example 1: Iron Hydroxide Sucrose Complex 1

The complex was prepared following the steps below.

1.1. Dissolving 75 g of FeCl₃·6H₂O in water to obtain 500 g of a ferric chloride solution with a mass percentage of 15%,

1.2. Mixing 45 g of sodium carbonate in water to obtain 450 g of a Na₂CO₃ aqueous solution with a mass percentage of 10%.

2. Preparation of Fe(OH)₃ suspension:

2.1. Adding the 10% Na₂CO₃ aqueous solution to the 15% ferric chloride solution and mixing evenly until the pH value of the mixture is 2.7. At this time, a large amount of carbon dioxide was generated and the color of the reaction solution changed from light brown to dark brown, which was still a clear solution.

2.2. Adding the 10% Na₂CO₃ aqueous solution to the clear solution and mixing evenly to obtain a reaction mixture having a pH value of 3.8. This was a crude iron hydroxide suspension. At this time, a large amount of particles precipitated from the solution.

2.3. Adding the remaining 10% Na₂CO₃ aqueous solution to the crude iron hydroxide suspension and mixing evenly to obtain an iron hydroxide suspension having a pH value of 5.

2.4. Collecting the pH 5 iron hydroxide suspension in a 10 L container, adding 9 L water under agitation, and allowing it to stand still. Removing the upper clear aliquot, then centrifuging the remaining mixture, adding water repeatedly during the centrifugation. Collecting the brown precipitate to obtain a polynuclear iron hydroxide, which had a chloride ion content of less than 0.05%.

3. Mixing in a 1 L container the polynuclear iron hydroxide thus obtained with 240 g of sucrose under agitation. The pH value of the resulting mixture was adjusted to 12 using a 20% sodium hydroxide solution. The reaction was carried out at 100° C. and pH 12 for 21 hours to obtain an iron hydroxide sucrose complex.

The molecular weight of the iron hydroxide sucrose complex thus obtained was determined by gel permeation chromatography (“GPC”) described below.

A. Instrument and Test Drug:

Agilent 1100 HPLC equipped with a Shodex differential refractive detector. Shodex P-82pullulan was used as the standard for molecular weights.

B. Method:

B.1 Chromatographic Conditions:

Chromatographic column: Waters Ultrahydrogel™ 7.8-mm×30 cm column with pore sizes of 1000 Å and 120 Å, respectively. Two columns were connected in series.

Mobile phase: phosphate buffer (containing 7.17 g disodium hydrogen phosphate dodecahydrate, 2.76 g disodium hydrogen phosphate, and 0.2 g sodium azide in 1000 ml of water).

Detector temperature: 45° C.

Column temperature: 45±2° C.

Flow rate: 0.5 ml.

Injection volume: 25 ul.

B.2 Sample Determination:

Preparing test solution by adding a predetermined amount of the sample to a mobile phase solution followed by filtration. Injecting 25 μl of the test solution for analysis. The data was processed with a GPC special software (HW-2000). The weight average molecular weight (Mw), the number average molecular weight (Mₙ), and D were calculated from a calibration curve obtained from the data generated from testing the standard under the same conditions.

B.3 GPC Spectrum of Test Results:

The molecular weight of the Shodex standard was 47100 Daltons.

The weight average molecular weight of the iron hydroxide sucrose complex of Example 1 was 46700 Daltons.

### Example 2: Iron Hydroxide Sucrose Complex 2

The complex was prepared as follows:

A.1. Preparing a 15% ferric chloride solution by dissolving 225 g of FeCl₃ 6H₂O in water to obtain 1500 g of the solution.

2. Dissolving 135 g sodium carbonate in water to obtain 1350 g of a Na₂CO₃ aqueous solution with mass fraction of 10%.

B. A polynuclear iron hydroxide suspension was prepared following the 3-step titration procedure described in Example 1 above using the ferric chloride solution and the Na₂CO₃ solution obtained in Step A.

C. Mixing the polynuclear iron hydroxide thus obtained with 720 g of sucrose in a 2 L container. The pH value of the resulting mixture was adjusted to 12 with a 20% sodium hydroxide solution, and then heating at 90° C. and pH 12 for 42 hours to obtain Iron Hydroxide Sucrose Complex 2.

### Example 3: Iron Hydroxide Sucrose Complex 3

The complex was prepared following a procedure similar to that described in Example 1 above.

A.1. A 15% ferric chloride solution (5000 g) was obtained by dissolving 750 g of FeCl₃·6H₂O in water.

2. A 10% Na₂CO₃ solution (4500 g) was obtained from 450 g sodium carbonate in water.

B. A polynuclear iron hydroxide was prepared following the 3-step titration procedure using the ferric solution and the Na₂CO₃ obtained in Step A above.

C. The polynuclear iron hydroxide was mixed with 2400 g of sucrose. The resulting mixture was adjusted to pH 12 with a 20% sodium hydroxide solution and heated for 32 h at 95° C. to obtain Iron Hydroxide Sucrose Complex 3, which had a pH value of 12.

### Evaluation

Iron Hydroxide Sucrose Complexes 1, 2, and 3 were evaluated for (a) free iron content (i.e., Fe³⁺ and Fe²⁺), (b) chloride ion content, and (c) reaction rate T75 against ascorbic acid.

(a) Detection of free iron (i.e., Fe³⁺ and Fe²⁺):

(1) Fe³⁺

A sample of a complex (5 mL) was mixed with 1 mL of 2 mol/L aqueous ammonia solution for 1 minute to observe whether there is brown precipitation. If so, free Fe³⁺ ions are present in the sample.

(2) Fe²⁺

(a) Potassium ferricyanide solution: weigh 1 g of potassium ferricyanide and add water until the resulting solution reaches 10 mL.

(b) Acetic acid sodium acetate buffer at pH 5.6: weigh 12 g of sodium acetate, dissolve it with 50 mL of distilled water, add 0.66 mL of acetic acid, and then adding water to 100 mL.

(c) A complex suspension (5 mL) was diluted to 50 mL with water to obtain an evaluation sample. Two testing solution were prepared, i.e., a blank solution and an evaluation solution.

In the blank solution was added 5 mL of the evaluation sample and 2 mL of acetic acid-sodium acetate buffer (pH 5.6).

In the evaluation solution was added 5 mL of the evaluation sample, 2 mL of acetic acid-sodium acetate buffer (pH 5.6), and 3 drops of potassium ferricyanide solution.

If the color of both solutions is the same, it is determined that there is no Fe₂₊ present in the complex suspension.

(b) Detection of Chloride Ion Concentration

Instruments and reagents: SSWY-810 rapid determination instrument of chloride ion content and the standard solution (0.005 mol/L and 0.0005 mol/L aq. NaCl). A chloride ion electrode and a glass electrode were calibrated before testing. The chloride ion concentration was registered by the electrodes.

(c) Determination of Reaction Rate T75 Against Ascorbic Acid

Reagents:
1. 0.9% sodium chloride solution as a diluting solution.
2. Vitamin C (ascorbic acid) stock solution: 8.8 g of vitamin C was mixed with water to prepare 50 mL stock solution.
3. Iron hydroxide sucrose complex stock solution: 15 mL of the iron hydroxide carbohydrate complex suspension from one of Examples 1-3 above was diluted to 50 mL with water.

Method:

All solutions above were maintained at 37° C. A testing solution included 20 mL of the NaCl solution, 4 mL of the vitamin C stock solution, and 1 mL of the complex stock solution.

The iron released from the complex was determined at 450 nm with a UV-vis spectrophotometer.

The iron content was calculated as:

100*[A(t)−A(n)/A(0)−A(n)],

wherein A(t) is the absorbance at time interval t minutes, A(n) is the background absorbance, and A(0) is the absorbance at time 0 (initial).

The results are shown in Table 1 below.

TABLE 1

| Iron hydroxide | | Free iron | reaction rate T75 |
|---|---|---|---|
| sucrose complex | Cl⁻ content, % | (Fe³⁺ and Fe²⁺) | (Minutes) |
| 1 | less than 0.025% | Not detected | 15 |
| 2 | less than 0.025% | Not detected | 13 |
| 3 | less than 0.025% | Not detected | 17 |

### Evaluation

Four samples, Stability Samples 1-4, were prepared and studied for stability for up to 3 months.

Stability Sample 1 contained in a sealed glass vial 5 mL of Iron Hydroxide Sucrose Complex 3 with its pH adjusted to 10.8 using a HCl solution. This sample was stored at 40° C.

Stability Sample 2 contained the same complex as Stability Sample 1 except that it was sterilized by autoclaving before stored at 40° C.

Stability Sample 3 contained in a sealed glass vial 5 mL of Iron Hydroxide Sucrose Complex 3 with its pH adjusted to 7 using a HCl solution. This sample was stored at 40° C.

Stability Sample 4 contained the same complex as Stability Sample 3 except that it was sterilized by autoclaving before stored at 40° C.

Molecular weights were analyzed at the end of each month. Results are shown in Table 2 below.

TABLE 2

| Stability Sample | Month 1 | Month 2 | Month 3 |
|---|---|---|---|
| 1 | 47121 | 47050 | 47178 |
| 2 | 47086 | 47144 | 47097 |
| 3 | 47086 | 47113 | 47150 |
| 4 | 46993 | 47098 | 47116 |

The results show that the molecular weights were little changed after stored for 3 months at 40° C., indicating that the iron hydroxide sucrose complexes are stable and can be sterilized at a high temperature under a high pH or neutral pH condition.

### Examples 4-8: Iron Hydroxide Carbohydrate Complexes 4-8

The complexes were prepared by the following steps: (1) diluting the purified polynuclear iron hydroxide suspension of Example 1 with the same amount of water, (2) mixing the diluted suspension with a carbohydrate to obtain a carbohydrate mixture, (3) adjusting the pH value of the carbohydrate mixture to 10 using a 20% sodium hydroxide solution, and (4) heating the pH-adjusted carbohydrate mixture at for 1 h to obtain an iron hydroxide carbohydrate complex as a product.

In Example 4, Iron Hydroxide Carbohydrate Complex 4, i.e., iron hydroxide erythritol complex, was obtained using erythritol as the carbohydrate with the ratio of iron:erythritol being 45:10.5.

In Example 5, Iron Hydroxide Carbohydrate Complex 5, i.e., iron hydroxide maltodextrin complex, was obtained using maltodextrin DE 30-35 as the carbohydrate with the ratio of iron:maltodextrin DE 30-35 being 30:50.

In Example 6, Iron Hydroxide Carbohydrate Complex 6, i.e., iron hydroxide maltodextrin glucose complex, was obtained using maltodextrin DE 30-35 and glucose (maltodextrin DE 30-35:glucose=9:1) as the carbohydrate with the ratio of iron:carbohydrate being 60:20.

In Example 7, Iron Hydroxide Carbohydrate Complex 7, i.e., iron hydroxide maltose complex, was obtained using maltose syrup (DE 58.5:glucose=1:1) as the carbohydrate with the ratio of iron:maltose syrup being 45:18.

In Example 8, Iron Hydroxide Carbohydrate Complex 8, i.e., iron hydroxide sorbitol complex, was obtained using sorbitol as the carbohydrate with the ratio of iron:sorbitol being 45:12.

FIG. 2 shows the structural diagram of a nanoparticle present in the iron hydroxide carbohydrate complexes prepared from the methods of this invention. The nanoparticle is a sphere-shaped iron carbohydrate colloid. In FIG. 2, a represents the polynuclear iron hydroxide core and b represents the carbohydrate shell coating the iron core. The carbohydrate shell has the following functions: 1. Stabilizing the iron hydroxide core, 2. maintaining the suspension of nanoparticles in water, 3. controlling the release of iron, 4. reducing the toxicity of iron, and 5. improve the taste of iron hydroxide product, i.e., eliminating an undesirable metallic taste.

### Examples 9-13: Iron Hydroxide Carbohydrate Complex Powders 9-13

Each of Iron Hydroxide Carbohydrate Complexes 4-8 was spray dried to obtain a powder from of the product.

Therefore, Example 9 is a powder of iron hydroxide erythritol complex of Example 4;

Example 10 is a powder of iron hydroxide maltodextrin complex of Example 5;

Example 11 is a powder of iron hydroxide maltodextrin glucose complex of Example 6;

Example 12 is a powder of iron hydroxide maltose complex of Example 7; and

Example 13 is a powder of iron hydroxide sorbitol complex of Example 8.

### Evaluation

Iron Hydroxide Carbohydrate Complex Powders 9-13 were evaluated for their iron content, free Fe³⁺ and Fe²⁺ content, and chloride ion content using the assays described above.

The results are shown in Table 3 below.

TABLE 3

| | Iron Content | Free iron | |
|---|---|---|---|
| Example | % | (Fe³⁺ and Fe²⁺) | Cl⁻ content |
| 9 | 45 | Not detected | Less than 0.1% |
| 10 | 27 | Not detected | Less than 0.1% |
| 11 | 42.1 | Not detected | Less than 0.1% |
| 12 | 42.5 | Not detected | Less than 0.1% |
| 13 | 43.5 | Not detected | Less than 0.1% |

### Example 14: Ferric Citrate Complex

### Polynuclear Iron Hydroxide

Similar to the procedure described in Example 1, polynuclear iron hydroxide was prepared using a three-step pH titration method.

(1) Solution A (15,000) was prepared by dissolving 2,250 g of solid FeCl₃ 6H₂O in water (15 wt %). In a separate container, Solution B (13,500 g) was prepared by dissolving in water 1350 g of Na₂CO₃ (10 wt %). Solution B was added slowly at 25° C. to Solution A under agitation until the pH value reached 2.8. The resulting clear solution was allowed to equilibrate for 5 minutes or until all CO 2 bubbles were released.

(2) Solution B was added at 25° C. under agitation to the clear solution of step (1) until the pH value reached 3.8, during which iron hydroxide was precipitated. The viscosity increased significantly requiring vigorous agitation. The suspension was allowed to equilibrate for 8 minutes to obtain a crude iron hydroxide suspension.

(3) To the crude iron hydroxide suspension was added at 25° C. under agitation the remaining of Solution B. Upon completion, the resultant crude polynuclear iron hydroxide suspension was allowed to equilibrate for 10 minutes. An equal volume of water was added for the next step.

(4) The crude polynuclear iron hydroxide suspension thus obtained was purified by centrifugation and repeatedly washing with water until the conductivity of the aliquot remained unchanged. A purified polynuclear iron hydroxide wet cake was obtained by removing water after centrifugation.

### Ferric Citrate

The purified iron hydroxide (0.4 mol) was suspended in an equal volume of water and mixed with a solution of citric acid (0.14 mol) and sodium citrate (0.14 mol). The resulting mixture was heated a 55-65° C. for 15-30 minutes. The cloudy mixture turned to a clear dark red solution. The Tyndall effect was observed, indicating formation of a colloid, i.e., the ferric citrate complex nanoparticles dispersed evenly in water.

Optionally, the clear solution of the ferric citrate complex was spray dried to obtain a ferric citrate complex powder.

### Water Solubility

The ferric citrate powder thus obtained (1 g) was dispersed in 1 mL of water. The suspension was a clear solution with a dark red color, demonstrating a water solubility of at least 50 wt %. The density is about 1.4 g/mL.

Two commercial solid ferric citrate products were studied for their water solubility as comparison. Ferric citrate 1 was commercially available from Yuzon Biotechnology (Zhengzhou, China). Ferric citrate 2 was commercially available from KonTai Food Additive Company (Tianjin, China). Both commercially products (1 g) were dispersed in water (up to 100 mL). At the concentration of 1 wt %, both products were not completely dissolved, indicating a water solubility of less than 1 wt %.

The ferric citrate complex of this invention shows a high water solubility, indicating great bioavailability and making it suitable for high strength liquid products.

### pH Stability

The ferric citrate powder of this invention (1 g) was dispersed in 1 mL of water to obtain a clear solution. The pH value of the clear solution was adjusted to 7, 3.5, 2.5, or 1.5 using a 1N HCl(aq.). The solution remained clear in each of the pH values, showing stability in the pH of 1.5-7.

### Molecular Weight

The ferric citrate suspension thus obtained was analyzed for its molecular weight using the GPC method described above. It was found that its weight average molecular weight is around 35000-45000 Daltons. The ferric citrate product having this molecular weight is ideal for bioavailability and iron release rate.

### Thermostability

The ferric citrate suspension was heated at 90° C. for 6 hours to check its thermostability. There was no change in terms of its appearance, solution clarity, and GPC profile, providing that the ferric citrate product is thermally stable. This good thermal stability makes the product suitable for preparing a sterile injection product.

### Stability After Spray Drying

Two samples were compared to show whether spray drying change the ferric citrate complex nanoparticles. The first sample was a ferric citrate suspension obtained in the procedure above. The second sample was a ferric citrate suspension prepared by dissolving a spray dried ferric citrate complex powder in water to the same concentration as the first sample. The two samples were the same in terms of their appearance, solution clarity, and GPC profile. The results demonstrated that the ferric citrate complex solution of this invention maintains its quality after spray drying, a process not suitable for many commercial ferric products due to decreased solubility. Instead, organic solvent was used to precipitate solid ferric products. See, e.g., U.S. Pat. No. 7,674,780.

Fe³⁺ and Fe²⁺

The presence/absence of free Fe³⁺ and Fe²⁺ were tested on the freshly prepared ferric citrate complex suspension following the procedures described above. The results showed that the ferric citrate complex thus prepared is absent of free Fe³⁺ or Fe²⁺ ions.

Free Fe³⁺ or Fe²⁺ ions are incompatible with many ingredients in pharmaceutical or nutraceutical formulations. Further, they contribute to an unpleasant metallic taste.

Absent of Fe³⁺ or Fe²⁺ ions, the ferric citrate product of this invention is suitable to be formulated into pharmaceutical or nutraceutical formulations.

### Example 15: Ferric Citrate Pyrophosphate Complex

The procedure described in Example 14 was followed except that, instead of the citric acid/citrate solution, a citric acid (0.3 mol) and pyrophosphate (0.3 mol) solution was used to obtain the ferric citrate pyrophosphate complex.

### Example 16: Ferric Gluconate

The procedure described in Example 14 was followed except that, instead of a citric acid/citrate solution, a gluconate (0.4 mol) solution was used to obtain the ferric gluconate of this invention.

### Example 17: Taste Evaluation

The powder of iron hydroxide maltodextrin glucose complex (Example 11) was dispersed in the same amount of water to obtain a colloid. Water and flavor agents were added to the colloid to prepare testing samples containing iron at 1%, 5%, and 10% for detecting the aftertaste of free Fe³⁺ and Fe²⁺. It was found that no unwanted metallic aftertaste was present in the testing samples.

### Example 18: Treating Iron Deficiency Anemia

The powder of iron hydroxide maltodextrin glucose complex (Example 11) was formulated into an oral liquid formulation containing 22.5 mg of iron in 10 mL colloid solution (Fe=0.25 wt %). Five female patients were instructed to take the formulation daily by oral for certain days (see Table 4 below). Blood samples were analyzed to determine the amount of hemoglobin using a Mission Hemoglobin Analyzer. The results are shown in Table 4.

TABLE 4

| | | | Hemoglobin before | Hemoglobin after |
|---|---|---|---|---|
| | | | taking complex | taking complex |
| Patient | Dose/day | Days | g/dL | g/dL |
| 1 | 1 | 30 | 6.1 | 9.1 |
| 2 | 2 | 24 | 7.1 | 11.2 |
| 3 | 2 | 6 | 8.4 | 11.7 |
| 4 | 2 | 6 | 9.9 | 10.7 |
| 5 | 1 | 12 | 10.2 | 12 |

As shown in Table 4 above, the hemoglobin level in each use is significantly increased after taking the iron hydroxide carbohydrate complex of this invention.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, complexes structurally analogous to the complexes of this invention also can be made, screened for their efficacy in treating iron deficiency anemia.

1. A method of preparing an iron hydroxide product, the method comprising the steps of:
adding a first base solution to a solution of a ferric salt to obtain Mixture A having a pH value of 2.7-2.8,
adding a second base solution to Mixture A to prepare a crude iron hydroxide suspension having a pH value of 2.8-3.8, and
adding a third base solution to adjust the pH of the crude iron hydroxide suspension to 5-9, followed by purification and concentration, thereby obtaining a purified polynuclear iron hydroxide suspension containing polynuclear iron hydroxide.

2. The method of claim 1, wherein before adding the second base, Mixture A is allowed to equilibrate for 1 minute to 15 minutes; and, before adding the third base, the crude iron hydroxide suspension is allowed to equilibrate for 2 minutes to minutes.

3. The method of claim 1, wherein each of the first base solution, the second base solution, and the third base solution, independently, is added at a temperature between 15° C. and 50° C., preferably between 20° C. and 40° C., and more preferably between 22° C. and 27° C.

4. The method of claim 1, wherein the crude iron hydroxide suspension is purified by washing with water and concentrated by removing water to an iron hydroxide concentration of 3 wt % to 16 wt %, and the purified iron hydroxide suspension has a chlorine content of less than 1%, preferably less than 0.1%.

5. The method of claim 1, wherein each of the first base solution, the second base solution, and the third base solution, independently, is an aqueous solution of a carbonate salt selected from the group consisting of NaHCO₃, Na₂CO₃, (NH₄)₂CO₃, and K₂CO₃.

6. The method of claim 5, wherein each of the first base solution, the second base solution, and the third base solution is an aqueous solution of Na₂CO₃ having a mass percentage of 1% to 25%, preferably 3% to 20%, more preferably 5% to 15%.

7. The method of claim 1, wherein the ferric salt is selected from the group consisting of Fe₂(SO₄)₃, Fe(NO₃)₃, and FeCl₃.

8. The method of claim 1, wherein the ferric salt is FeCl₃ having a mass percentage of 5% to 60%, preferably 15% to 25%.

9. The method of claim 1, further comprising the steps of:
mixing the purified polynuclear iron hydroxide suspension and a carbohydrate to obtain a carbohydrate mixture,
adjusting the pH value of the carbohydrate mixture to 7.5-13 or 10-13.5, and
heating the pH-adjusted carbohydrate mixture to a temperature of 60° C.-125° C., preferably 80° C.-95° C., thereby producing an iron hydroxide-carbohydrate complex suspension, the mass ratio between iron and carbohydrate being (1-1100):100,
wherein the iron hydroxide product is the iron hydroxide-carbohydrate complex.

10. The method of claim 9, wherein the carbohydrate is selected from the group consisting of monosaccharides, disaccharides, oligosaccharides, polysaccharides, hydrolyzed polysaccharides, and any combinations thereof.

11. The method of claim 9, wherein the pH value of the carbohydrate mixture is adjusted by adding a fourth base that is a hydroxide solution selected from the group consisting of a NH₄OH solution, a KOH solution, and a NaOH solution, and the hydroxide solution has a mass percentage of 5% to 50%, preferably 10% to 25%.

12. The method of claim 9, wherein the pH value of the carbohydrate mixture is adjusted to 10-13.5 and the pH-adjusted carbohydrate mixture is heated at 80° C.-125° C. and preferably 85° C.-95° C. for 1 hour to 50 hours.

13. The method of claim 9, wherein the carbohydrate is sucrose and the mass ratio between Fe³⁺ and sucrose is 1:(10-20), preferably 1:(13-17).

14. The method of claim 9, wherein the pH value of the iron hydroxide-carbohydrate complex suspension is adjusted, using a pH modifier, to 5.5-11.1, preferably 10.5-11.2, more preferably 6.5-7.5.

15. The method of claim 14, wherein the pH modifier is HCl, NaOH, citric acid, oxalic acid, fumaric acid, tartaric acid, succinic acid, malic acid, ascorbic acid, phosphoric acid, pyrophosphoric acid, or glycophosphoric acid.

16. The method of claim 9, further comprising the step of drying the iron hydroxide-carbohydrate complex suspension.

17. The method of claim 9, wherein the iron hydroxide-carbohydrate complex has a weight average molecular weight of 30000-60000 Daltons, and has a reaction rate T75 against ascorbic acid of 35 minutes or less, and is absent of free ferric ions.

18. The method of claim 9, wherein the pH value of the carbohydrate mixture is adjusted to 7.5-13 and the pH-adjusted carbohydrate mixture is heated for 0.2 hours to 30 hours.

19. The method of claim 18, wherein the pH value of the carbohydrate mixture is adjusted to 9-12.5, the pH-adjusted carbohydrate mixture is heated at a temperature of 65° C.-121° C., preferably 80° C.-95° C., for 0.2 hours to 30 hours, and the mass ratio between iron and carbohydrate is (1-264):24.

20. The method of claim 9, wherein the iron hydroxide-carbohydrate complex, having a water solubility of wt % or more, contains, by dry weight, iron 10%-47% and chloride ion less than 1%, preferably less than 0.1%.

21. The method of claim 9, further comprising the step of formulating the iron hydroxide-carbohydrate complex into drops, oral liquid, suspension, injection, powder, capsule, tablet, or lozenge for treating iron deficiency anemia in humans or animals.

22. The method of claim 1, further comprising the steps of:
mixing the purified polynuclear iron hydroxide suspension with citric acid, a citrate salt, or combination thereof to obtain a citrate mixture, and
heating the citrate mixture at a temperature of 45° C.-95° C., preferably 55° C.-65° C. for 2 minutes 180 minutes, preferably 5 minutes-30 minutes, thereby producing an ferric citrate suspension containing iron hydroxide-citrate complex, the molar ratio between iron and citrate being 1:(0.3-5), preferable 1:(0.6-1.5),
wherein the iron hydroxide product is an iron hydroxide-citrate complex.

23. The method of claim 22, wherein the iron hydroxide-citrate complex, having a water solubility of 20 wt % or greater, preferably 50 wt % or greater, contains by dry weight iron 5%-35%, preferably 12%-25% and is absence of free ferric ion.

24. The method of claim 22, further comprising the step of drying the ferric citrate suspension to a dry iron hydroxide-citrate complex.

25. The method of claim 24, further comprising the step of formulating the dry iron hydroxide-citrate complex into drops, oral liquid, suspension, injection, powder, capsule, tablet, or lozenge for treating iron deficiency anemia in humans or animals.

26. The method of claim 1, further comprising the steps of:
mixing the purified polynuclear iron hydroxide suspension and a solution containing (i) citric acid or a citrate salt and (ii) pyrophosphoric acid or a pyrophosphate salt to obtain a pyrophosphate mixture, and
heating the pyrophosphate mixture at a temperature of 55° C.-65° C. for 5 minutes to 10 hours, preferably 25-55 minutes, thereby producing a ferric citrate pyrophosphate suspension, the molar ratio of iron:citrate:pyrophosphate being 1:(0.3-3):(0.3-3),
wherein the iron hydroxide product is an iron hydroxide-citrate-pyrophosphate complex.

27. The method of claim 26, wherein the iron hydroxide-citrate-pyrophosphate complex, having a water solubility of 20 wt % or greater, preferably 50 wt % or greater, contains by dry weight iron 3%-35%, preferably 5-20%.

28. The method of claim 1, further comprising the steps of:
mixing the purified polynuclear iron hydroxide suspension with a carboxylated carbohydrate, or a mixture thereof to obtain a carboxylated carbohydrate mixture, and
heating the carboxylated carbohydrate mixture at a temperature of 65° C.-125° C. preferably 65° C.-75° C. for 5 minutes to 10 hours, preferably 25 minutes to 55 minutes, thereby producing a ferric carboxylated carbohydrate suspension, the molar ratio between iron and the carboxylated carbohydrate being 1:(0.3-5), preferable 1:(0.5-1.5), wherein the iron hydroxide product is an iron hydroxide-carboxylated carbohydrate.

29. The method of claim 28, wherein the carboxylated carbohydrate is gluconate.

30. The method of claim 1, further comprising the steps of:
mixing the purified polynuclear iron hydroxide suspension with a multivalent anion to obtain a multivalent anion mixture,
adjusting the pH value of the multivalent anion mixture to 2-13, preferably 3-9, and heating the pH-adjusted multivalent anion mixture to a temperature of 40° C.-125° C., preferably 50° C.-95° C., thereby producing a nano ferric complex suspension, the mass ratio between iron and the multivalent anion being (1-1100):100,
wherein the iron hydroxide product is the nano ferric complex.

31. An iron hydroxide product prepared by the method of claim 1.
